(19) United States
(12) Patent Application Publication
(10) Pub. No.: US 2009/0220982 A1
(43) Pub. Date: Sep. 3, 2009

(54) COMPOSITIONS AND METHODS FOR DETERMINING NEPHROTOXICITY

(75) Inventors: Eliana Saxon Armstrong, Oakland, CA (US); Lee Ann Feeney, San Francisco, CA (US); Corwin F. Kostrub, Pacifica, CA (US)

Correspondence Address: SEED INTELLECTUAL PROPERTY LAW GROUP PLLC, 701 FIFTH AVE, SUITE 5400, SEATTLE, WA, 98104, US

(73) Assignee: Achaogen Inc.., South San Francisco, CA (US)

(21) Appl. No.: 12/392,035

(22) Filed: Feb. 24, 2009

Related U.S. Application Data
(60) Provisional application No. 61/032,745, filed on Feb. 29, 2008.

(51) Int. Cl. C12Q 1/02; C12Q 1/34; C12Q 1/26; C12Q 1/68

(52) U.S. Cl. 435/6; 435/29; 435/18; 435/25

(57) ABSTRACT

The present invention provides novel in vitro assays for determining the nephrotoxicity of a compound. These assays correlate well with in vivo nephrotoxicity and also provide high-throughput methods to screen multiple compounds for in vivo nephrotoxicity. In addition, the methods of the present invention may be adapted to screen for nephroprotectant compounds, including those that protect cells and animals from the nephrotoxic effects of aminoglycoside antibiotics.

### CROSS-REFERENCE TO RELATED APPLICATION

This application claims the benefit under 35 U.S.C. § 119(e) of U.S. Provisional Patent Application No. 61/032,745 filed Feb. 29, 2008, where this provisional application is incorporated herein by reference in its entirety.

### BACKGROUND

1. Technical Field

The present invention is directed to methods of determining the nephrotoxicity of compounds and methods of identifying nephroprotectants.

2. Description of the Art

Numerous drugs and other substances are known to be nephrotoxic and can cause renal failure through a variety of mechanisms including direct toxicity to the renal tubules, allergic interstitial nephritis, and crystallization of the drug within the renal tubules. Nephrotoxic drugs include anticancer agents such as cisplatin, methotrexate, and doxyrubicin; non-steroidal anti-inflammatories (NSAIDS) (e.g., COX-2 inhibitors), antivirals (e.g., acyclovir, indinivir), acetylcholinesterase inhibitors, angiotensin II receptor blockers (ARBs), lithium, radiographic contrast media, and antibiotics (e.g., aminoglycosides, amphotericin).

Aminoglycoside antibiotics are the most commonly used antibiotics worldwide in the treatment of Gram-negative bacterial infections. However, aminoglycosides induce nephrotoxicity in 10-20% of therapeutic courses. Several in vitro approaches have been used to evaluate aminoglycoside toxicity. These assays have evolved as the mechanistic understanding of aminoglycoside toxicity has developed. However, the critical interactions underlying these mechanisms are complex and remain under investigation (Mingeot-Leclerq et al., 1999). Without a clear consensus on the molecular mechanism of aminoglycoside toxicity, devising an assay that employs a single sub-cellular component is both important and challenging.

Binding of a positively charged aminoglycoside to negatively charged membrane phospholipids is thought to be part of the entry process into both kidney proximal tubule cells and inner ear hair cells (Humes et al., 1992, Sastrasinh et al., 1982). Schacht and coworkers used a model phospholipid bilayer with a non-covalently associated fluorescent probe to monitor binding of aminoglycosides (Au et al., 1986). This assay was specific for phosphatidylinositol bisphosphate and was successfully used to rank the several aminoglycoside toxicities in accordance with their previously reported ototoxicity. Unfortunately, the technique requires extensive purification of the samples and a dust-free environment, which limits its utility as a screening platform.

Another likely player in the initial uptake of aminoglycosides into target cells is megalin (Nagai et al., 2001). Megalin is a large receptor glycoprotein for multiple physiologically important ligands, including vitamins and hormones. Megalin expression is observed at the primary sites of aminoglycoside toxicity (kidney and inner ear), as well as in several other tissues. A key piece of evidence implicating megalin in aminoglycoside uptake is that mice deficient in megalin function accumulate significantly lower levels of gentamicin in the kidney (Schmitz et al., 2002). The development of renal cell lines with stable megalin expression was an initial example of cell-based aminoglycoside nephrotoxicity assays that were more versatile than animal models and potentially more relevant than in vitro cell-free systems (Nielsen et al., 1998).

One example of a stable cell line expressing megalin is the porcine kidney tubule cell line, LLC-PK1. The LLC-PK1 cell line has formed the basis of several cell based nephrotoxicity assays. When grown to confluence in cell culture, tight junctions are formed, and the cells begin unidirectional transport of water and salts, which results in the formation of “domes”. Domes are sections of the monolayer that separate from the culture dish and are visible using light microscopy. These domes indicate healthy, functioning LLC-PK1 cells, and therefore, dome loss is one measure of the extent of aminoglycoside toxicity. This toxicity readout was validated by the discovery that a series of aminoglycosides with different nephrotoxic potential in the rat model showed the same rank order in their ability to degrade domes in LLC-PK1 culture (Takamoto et al., 2002). Although the initial results with the LLC-PK1 dome loss assay showed correlation with in vivo data of aminoglycoside activity, the assay requires careful microscopic analysis of each sample, and thus, this labor intensive method is not suitable for screening large numbers of novel compounds.

Another LLC-PK1 assay was reported by Mingeot-Leclercq and coworkers (Servais et al., 2005). Cultured LLC-PK1 cells were treated with gentamicin, and the resulting apoptosis response was measured by various spectrophotometric and staining methods. This more direct, quantitative approach provided the opportunity to observe a dose-response in gentamicin-induced toxicity, which may be an important capability when attempting to distinguish closely related compounds. Subsequently, the same group demonstrated that gentamicin caused a dose-dependent apoptosis response at much lower gentamicin concentrations when the aminoglycoside was introduced to the LLC-PK1 cells by electroporation (Servais et al., 2006). However, this electroporation technique does not measure the toxicity of aminoglycosides via their in vivo uptake route, which could lead to an inaccurate estimation of their in vivo nephrotoxicity.

Drug-associated nephrotoxicity may be treated or prevented by the administration of nephroprotectant compounds. Nephroprotectants are compounds that ameliorate the toxicity induced by aminoglycosides and other nephrotoxic compounds. An extensive body of literature describes such candidate nephroprotectant compounds that afford protection against aminoglycoside antibiotics. A non-limiting example of a list of nephroprotectants includes aspirin, benzoic acid derivatives, poly-phenols, and poly-anions. Much of the work testing candidate nephroprotectants has been performed in animal models. However, using animal models limits the number of compounds that can be studied and is not suitable for screening large numbers of compounds.

One example of a well studied aminoglycoside nephroprotectant in human clinical trials is aspirin. Patients treated concurrently with intravenous gentamicin and oral aspirin suffered significantly less hearing loss than a similar group of patients treated with gentamicin alone (Sha et al., 2006). However, the aspirin treatment caused gastric bleeding in some patients, which resulted in their removal from the study. Even with this side effect, the toxicity prevention observed with aspirin treatment may be useful, given that gentamicin-induced ototoxicity can be debilitating and is generally irreversible. Aspirin is thought to modulate aminoglycoside toxicity by acting as an anti-oxidant and reversing the effects of reactive oxygen species (ROS) generated by aminoglycoside exposure (Chen et al., 2006). However, aspirin does not seemed extremely well suited for use as a nephroprotectant, because it can cause both acute and chronic nephrotoxicity at high doses in humans and experimental animals (Black 1986).

Additional examples in the art of aminoglycoside nephroprotectants include other benzoic acid derivatives. One of these compounds, 2,3-dihydroxybenzoate (DHB), has shown a protective effect in both aminoglycoside ototoxicity and nephrotoxicity animal models. In an ototoxicity animal model, Song and colleagues found that DHB was an effective co-therapy administered with gentamicin, which resulted in significantly lower shifts in auditory thresholds in guinea pigs (Song et al., 1996). In a rat nephrotoxicity model, treatment with DHB reduced elevations in blood, urea, nitrogen (BUN) and serum creatinine after gentamicin treatment for eight days (Walker et al., 1998). Unlike aspirin, DHB appears to present a benign toxicity profile. As part of a DHB safety and efficacy study for iron chelation treatment of β-thalassemia, patients were treated with oral doses of DHB for one year (Miller 1979). Although no toxicity was observed, DHB did not exhibit the desired therapeutic efficacy and was not pursued further.

Studies performed by Graziano and colleagues revealed that DHB inhibits H₂O₂ induced membrane peroxidation in erythrocytes, adding further support to the proposal that ROS play a role in aminoglycoside toxicity (Graziano et al., 1976). Additional studies confirmed that DHB treatment provided only partial protection from gentamicin nephrotoxicity in rats. In addition, DHB has poor solubility in aqueous solution, which significantly limits its utility as a co-therapy with aminoglycosides.

Polyphenolic natural products such as the putative anti-oxidants resveratrol and curcumin have been investigated as nephroprotectants (Silan et al., 2007; Farombi et al., 2006). In rat studies, these compounds only attenuated gentamicin toxicity but did not prevent it. The majority of compounds in this class have limited utility as aminoglycoside nephroprotectants due to their poor aqueous solubility.

Lysozomal sequestration of aminoglycosides affords protection from nephrotoxicity in rats (Williams et al., 1986). In particular, poly-anions and polyaspartic acid have been shown to prevent aminoglycoside binding to renal brush border membranes in vitro (Kishore et al., 1990; Kishore et al., 1992). The interaction between positively charged aminoglycosides and negatively charged polypeptides leads to lysozomal accumulation of aminoglycosides. This mechanism may be more effective than radical-scavenging, as it interferes with the aminoglycoside toxicity response at an earlier stage. However, an important caveat to using polyaspartic acid to direct the lysozomal accumulation of aminoglycosides may be that nephrotoxicity is merely delayed, rather than prevented. At the end of a 14 day treatment period with gentamicin and/or polyaspartic acid, rats co-dosed with polyaspartic acid showed a five-fold higher gentamicin concentration in their kidneys compared to rats treated with gentamicin alone. However, after the two week treatment period, subsequent monitoring of the rats revealed that the all of the gentamicin was eliminated from the kidney over the following 16 weeks, with no creatinine elevation or histopathological changes in the co-dosed polyaspartic acid treated animals. These results support the use of polyaspartic acid as a nephroprotectant against aminoglycosides. Studies in both rats and dogs have suggested that co-administration of polyaspartic acid may not interfere with aminoglycoside antibiotic therapy (Reinhard et al., 1994; Whittem et al., 1996).

In summary, while a variety of nephroprotectant compounds exist in the art, many of these have proven to be of limited effectiveness in preventing human nephrotoxicity and are associated with undesirable side effects. Thus, there is a clear need for safe and effective nephroprotectant compounds suitable for human use. Additionally, given the significant dangers associated with the nephrotoxicity of numerous drugs, there clearly remains a need to be able to determine the nephrotoxicity of therapeutic compounds in order to safely treat human infections and disease.

The present invention addresses these needs by providing novel high-throughput methods for determining the nephrotoxicity of compounds as well as for identifying novel nephroprotectant compounds. These in vitro methods for determining compound nephrotoxicity correlate well with in vivo nephrotoxicity, and are, therefore, suitable predictors of in vivo nephrotoxicity.

### BRIEF SUMMARY

The present invention is based on the development of a novel in vitro assay of nephrotoxicity, which may be used to determine in vivo nephrotoxicity of compounds and identify nephroprotectants.

In one embodiment, the present invention includes a method for determining the nephrotoxicity of one or more test compounds, said method comprising: (i) contacting discrete populations of human kidney epithelial cells with one or more test compounds; wherein each of a plurality of different concentrations for each of said one or more test compounds contacts a separate discrete population of cells; (ii) determining the level of an indicator of nephrotoxicity for each of said populations of contacted cells of step (i) in order to produce a dose response curve for each of the one or more test compounds; and (iii) determining the nephrotoxicity of each of said one or more test compounds. In one embodiment, the cells are human, and in a related embodiment, the cells are HK-2 cells.

In one embodiment, the present invention includes a method for determining the nephrotoxicity of one or more test compounds or a combination of test compounds, said method comprising: (i) contacting discrete populations of human kidney epithelial cells with one or more test compounds or compound mixtures; wherein each of a plurality of different concentrations for each of said one or more test compounds or compound mixtures contacts a separate discrete population of cells; (ii) determining the level of an indicator of nephrotoxicity for each of said populations of contacted cells of step (i) in order to produce a dose response curve for each of the one or more test compounds; and (iii) determining the nephrotoxicity of each of said one or more test compounds. In one embodiment, the cells are human, and in a related embodiment, the cells are HK-2 cells.

In related embodiments, each of the populations of cells is located in separate wells in a tissue culture device comprising a plurality of wells. In particular embodiments, the plurality of wells is selected from the group consisting of 4, 6, 12, 24, 48, 96, 384, and 1536 wells.

In another embodiment, the above method for determining the nephrotoxicity of one or more test compounds, further comprises: (iv) contacting additional discrete populations of kidney epithelial cells with a plurality of concentrations of one or more control nephrotoxic compounds; (v) determining the level of an indicator of nephrotoxicity for each of said additionally contacted populations of cells of step (iv), in order to produce a dose response curve for each of said one or more control nephrotoxic compounds in said additionally contacted populations of cells of step (iv); and (vi) determining the nephrotoxicity of each of the one or more control nephrotoxic compounds.

In related embodiments, the plurality of concentrations of the one or more test compounds tested and the plurality of concentrations of the one or more control nephrotoxic compounds tested are both in the range of about 1 mg/mL to about 1 ug/mL.

In another related embodiment, the determination of the nephrotoxicity of each of said one or more compounds comprises calculating an EC50 for each of the one or more test compounds from the dose response curves of step (ii).

In further related embodiments, the determination of the nephrotoxicity of each of said one or more compounds comprises comparing the EC50 for each of the one or more test compounds to an EC50 for each of the one or more nephrotoxic compounds calculated from the dose response curves from step (iv).

In yet other related embodiments, the one or more control nephrotoxic compounds are selected from the group consisting of: amikacin, gentamicin, kanamycin, neomycin, netilmicin, paromomycin, streptomycin, tobramycin, apramycin.

In particular embodiments, the one or more test compounds are aminoglycosides.

In some embodiments, the indicator of nephrotoxicity is apoptosis.

In further embodiments, the indicator of nephrotoxicity is caspase activity.

In yet other embodiments, the caspase activity is caspase-3 activity.

In specific embodiments, the caspase-3 activity is determined using a bioluminescent substrate.

A further related embodiment of the present invention is a method for determining the in vivo nephrotoxicity of a test compound, said method comprising: (i) determining the EC50 of the test compound and one or more control nephrotoxic compounds using an in vitro kidney epithelial cell assay; (ii) comparing the EC50 of the test compound to the EC50 of one or more control nephrotoxic compounds, wherein the EC50 of the one or more in vivo nephrotoxic compounds correlates with an in vivo indicator of nephrotoxicity, thereby determining the in vivo nephrotoxicity of a test compound.

In related embodiments, the in vivo indicator of nephrotoxicity is selected from the group consisting of: an increase in BUN levels, increased serum creatinine levels, and caspase activity.

In further related embodiments, the in vivo indicator of nephrotoxicity is an increase in BUN levels.

In another embodiment, the present invention includes a method for determining the ability of one or more candidate nephroprotectant compounds to act as a nephroprotectant, said method comprising: (i) contacting discrete populations of HK-2 cells with a plurality of different concentrations of one or more candidate nephroprotectant compounds in the presence of a nephrotoxic compound, wherein each of a plurality of different concentrations for each of said one or more candidate nephroprotectant compounds contacts a separate discrete population of HK-2 cells; (ii) determining the levels of an indicator of nephrotoxicity for each one of the contacted populations of HK-2 cells of step (i); and (iii) determining the ability of each one or more candidate nephroprotectant compounds to act as a nephroprotectant.

In a further related embodiment, the above method further comprises: (iv) contacting additional discrete populations of HK-2 cells with a plurality of different concentrations of said one or more candidate nephroprotectant compounds in the absence of a nephrotoxic compound, wherein each of a plurality of different concentrations for each of said one or more candidate nephroprotectant compounds contacts a separate discrete population of HK-2 cells, wherein the ability of a candidate nephroprotectant compound to act as a nephroprotectant is determined when a dose-dependent decrease in the indicator of nephrotoxicity is present in the contacted HK-2 cells of step (i) compared to the indicator of nephrotoxicity in the contacted HK-2 cells of step (iii) for a given candidate nephroprotectant compound.

In other embodiments, the present invention includes a method for determining the ability of one or more candidate nephroprotectant compounds to act as a nephroprotectant, said method comprising: (i) contacting discrete populations of kidney epithelial cells with a plurality of different concentrations of said one or more candidate nephroprotectant compounds, wherein each of a plurality of different concentrations for each of said one or more test compounds contacts a separate discrete population of cells; (ii) contacting additional discrete populations of cells with a plurality of different concentrations of said one or more candidate nephroprotectant compounds as in step (i), and further contacting all the additional discrete populations of cells with a static concentration of nephrotoxic compound; (iii) determining the levels of an indicator of nephrotoxicity for each one of the contacted populations of cells of step (i) and step (ii); and (iv) determining the ability of each one or more candidate nephroprotectant compounds to act as a nephroprotectant.

In related embodiments, a dose-dependent decrease present in the indicator of nephrotoxicity in the contacted cells of step (iii) compared to the indicator of nephrotoxicity in the contacted cells of step (ii) for a given candidate nephroprotectant compound; and wherein the contacted cells of step (ii) and step (iii) have the same concentration of the given candidate nephroprotectant compound determines the given candidate nephroprotectant compound's ability to act as a nephroprotectant.

In some embodiments, the indicator of nephrotoxicity is apoptosis. In particular embodiments, the indicator of nephrotoxicity is caspase activity. In further embodiments, the caspase activity is caspase-3 activity.

In another embodiment, the method for determining the ability of one or more candidate nephroprotectant compounds to act as a nephroprotectant further comprises: (v) validating the ability of said one or more candidate nephroprotectant compounds to act as a nephroprotectant by performing one or more counterscreens.

In further embodiments, said one or more counterscreens are selected from the group consisting of: a cell viability assay, and an assay for luciferase activity. In particular embodiments, the counterscreens are a cell viability assay followed by and an assay for luciferase activity. In another particular embodiment, the luciferase activity is produced in the presence of viable cells.

In certain embodiments a decrease in the number of cells in the cell viability assay and/or a decrease in the luciferase activity determines that the one or more candidate nephroprotectant compounds are not nephroprotectants.

In some embodiments, the one or more candidate nephroprotectant compounds are selected from the group consisting of: antioxidants, compounds that structurally resemble antioxidants, peptides, peptidomimetics, amino acids, amino acid analogs, polynucleotides, polynucleotide analogs, nucleotides, nucleotide analogs, and organic, inorganic compounds, and any combination thereof.

In yet another related embodiment, the present invention includes kits for determining the nephrotoxicity of a candidate nephrotoxic compound comprising: (i) instructions for using the kit; (ii) a multi-well culture vessel of about 24, 48, 96, 384, or 1536 wells; (iii) a luciferase substrate specific for caspase activity; and (iv) a control nephrotoxic compound. In related embodiments the kits include kidney epithelial cells, e.g., HK-2 cells. In other related embodiments, the candidate nephrotoxic compound is selected from the group consisting of aminoglycosides and aminoglycoside antibiotics.

In a further embodiment, the present invention also includes kits for determining the ability of a candidate nephroprotectant compound to act as a nephroprotectant comprising: (i) instructions for using the kit; (ii) a multi-well culture vessel of about 24, 48, 96, 384, or 1536 wells; (iii) a luciferase substrate specific for caspase activity; (iv) a control nephrotoxic compound; (v) a cell viability determining reagent; and (vi) luciferase conditional on cell viability. In related embodiments, the kits further comprise kidney epithelial cells, e.g., HK-2 cells.

In other related embodiments, the candidate nephroprotectant compound is selected from the group consisting essentially of: antioxidants, compounds that structurally resemble antioxidants, peptides, peptidomimetics, amino acids, amino acid analogs, polynucleotides, polynucleotide analogs, nucleotides, nucleotide analogs, and organic, inorganic compounds, and any combination thereof.

### BRIEF DESCRIPTION OF THE SEVERAL VIEWS OF THE DRAWINGS

FIG. 1 is a schematic outline of the HK-2 in vitro nephrotoxicity assay. The compound corresponding to the right-most line in the graph is predicted to be more nephrotoxic than the compound corresponding to the left-most line.

FIG. 2 is a graph showing results obtained for amikacin and gentamicin in the HK-2 in vitro nephrotoxicity assay. Compounds were run in duplicate with the high and the low values indicated as error bars.

FIG. 3 provides graphs comparing the correlation of different in vitro nephrotoxicity assays with the in vivo nephrotoxicity of various compounds. FIG. 3A is a graph showing a significant correlation between the results of the HK-2 in vitro nephrotoxicity assay and the in vivo 14-day rat study conducted with amikacin, apramycin, gentamicin, neomycin, compound A, and compound B. FIG. 3B is a graph showing poor correlation between the results of the LLC-PK1 in vitro nephrotoxicity assay and the in vivo 14-day rat study conducted with amikacin, apramycin, gentamicin, neomycin, compound A, and compound B.

FIG. 4 is a graph showing a correlation between apoptosis readout of an in vitro LLC-PK1 nephrotoxicity assay for a group of test compounds and the relative nephrotoxicity of these compounds established in a 14-day rat study.

FIG. 5 is a graph showing the results of an in vivo HK-2 nephrotoxicity assay performed in the presence of various concentrations of an aminoglycoside-dependent candidate nephroprotectant compound, in the presence or absence of the aminoglycoside. The results show concentration dependent reduction in the gentamicin-induced caspase signal.

FIG. 6 is a flowchart of counterscreens used to evaluate potential nephroprotectants.

### DETAILED DESCRIPTION

The present invention is based, in part, upon the identification of an in vitro assay for determining the nephrotoxicity of a compound. As used herein, the term “nephrotoxic” describes a compound or an effect of a compound that is damaging or toxic to the kidney. Nephrotoxic injury can lead to acute renal failure, in which the kidneys suddenly lose their ability to function, or chronic renal failure, in which kidney function slowly deteriorates.

The results obtained using the assays described herein correlate well with in vivo indicators of nephrotoxicity. Thus, the present in vitro assay may be used to determine or predict the in vivo nephrotoxicity of a compound. Moreover, the level of in vitro nephrotoxicity for compounds assayed by the methods described herein correlates well with the relative in vivo nephrotoxicity among those compounds. In addition, the present assays are amenable to high-throughput methods, and may be performed using automated devices to evaluate multiple test compounds simultaneously.

In various embodiments, the methods of the present invention may, therefore, be utilized to determine the in vitro or in vivo nephrotoxicity of one or more compounds, to predict the in vivo nephrotoxicity of one or more compounds, to predict the relative in vivo nephrotoxicity among a group of compounds, and to screen one or more compounds to identify nephrotoxic compounds.

One of skill in the art would appreciate that the nephrotoxicity for mixtures of compounds can be different from or the same as the aggregate individual compound nephrotoxicity. Thus, as the therapeutic benefits achieved from mixtures of compounds (e.g., aminoglycosides) can outweigh the combined nephrotoxicity of said mixtures, embodiments of the present invention are suitable to determine the nephrotoxicity of a compound mixture.

Many of the previous studies in the art have examined the nephrotoxicity of compounds in a piecemeal fashion with a variety of dissimilar assays. The present invention addresses the lack of standard format, large scale methods for determining the nephrotoxicity of test compounds. By rapidly screening a plurality of test compounds using the in vitro methods of the present invention, the skilled artisan can readily identify more suitable therapeutic compounds (i.e., with lower indices of nephrotoxicity), which may be chosen to safely and effectively treat human disease and infection.

This is particularly relevant for the identification of aminoglycoside antibiotics. As used herein, the term “aminoglycoside” means an antibiotic compound that is characterized by the presence of an aminocyclitol ring linked to an aminosugar in its structure. This group of antibiotics is effective against aerobic and facultative aerobic Gram-negative bacilli and some Gram-positive bacteria such as staphylococci. A non-limiting list of commonly known aminoglycosides includes: amikacin, gentamicin, kanamycin, neomycin, netilmicin, paromomycin, streptomycin, tobramycin, apramycin, and modified derivatives thereof. Aminoglycoside antibiotics are the most common form of treatment for gram negative bacterial infections in the world. Yet, the use of aminoglycoside antibiotics to treat such infections results in nephrotoxicity in 10-20% of the cases. Thus, high-throughput, in vitro assays designed to identify aminoglycoside antibiotics with relatively low inherent nephrotoxicity are beneficial in both treating bacterial infections and reducing the health care costs covering the treatment of kidney damage. Accordingly, in particular embodiments, compounds assayed by the methods of the present invention are aminoglycoside antibiotics.

In addition, in various embodiments, the methods of the present invention may be utilized to identify nephroprotectant compounds. As used herein, the term “nephroprotectant” means a compound or agent that is able to ameliorate, reduce, inhibit, or prevent the damaging or toxic effects induced by a nephrotoxic compound. Since the present assays may be performed in a high-throughput manner, the assays can be used to simultaneously measure the ability of one or more compounds to act as nephroprotectant compounds against a broad spectrum of nephrotoxic compounds or against specific nephrotoxic compounds, such as aminoglycosides. In various embodiments, the methods of present invention, therefore, may be used to screen for the ability of one or more compounds to act as a nephroprotectant; ameliorate, prevent, inhibit, or reduce the nephrotoxicity of a compound; and/or function as an inhibitor of caspase activity, thereby resulting in a nephroprotective effect.

One of skill in the art would understand that mixtures or combinations of compounds can provide a level of nephroprotection that is different from or the same as the levels of aggregate individual compound nephroprotection. Thus, in certain embodiments, methods of the present invention are suitable and used to determine the level of nephroprotection of a compound mixture.

In certain embodiments, methods of the present invention are practiced using HK-2 cells or LL-PK1 cells. HK-2 cells are a human kidney epithelial cell line, available from American Type Culture Collection (ATCC; CRL-2190). As described in the accompanying Examples, assays performed using these cells resulted in nephrotoxicity values that closely correlated with in vivo indicators of nephrotoxicity, while LLC-PK1 cells, a porcine kidney tubule cell line, produced nephrotoxicity values that correlated well with the rank order of in vivo nephrotoxicity among different compounds. Accordingly, in particular embodiments, cells utilized according to the present invention are any kidney epithelial cells or kidney tubule cells. These cells may be derived from primary cells or may be from a cell line. The cells may be obtained from any mammalian source that is amenable to primary culture and/or adaptation into cell lines. In lieu of generating cell lines from animals, such cell lines may be obtained from, for example, American Type Culture Collection, (ATCC, Rockville, Md.), or any other Budapest Treaty or other biological depository. In one embodiment, the cells are derived from humans or other primates, rats, mice, rabbits, sheep, dogs, and the like. In one preferred embodiment, the cells are human kidney epithelial cells. In certain embodiments, the cells are porcine kidney tubule cells.

Techniques employed in mammalian primary cell culture and cell line cultures are well known to those of ordinary skill in that art. Indeed, commercially available cell lines are generally accompanied by specific directions for culturing cells in preferred growth conditions, along with particular media formulations that are optimized for that given cell line. The cells may be cultured or grown in any suitable growth media. The media may optionally be serum-free. In one particular embodiment, the cells are cultured in media comprising one or more growth factors, such as, epidermal growth factor (EGF) and/or bovine pituitary extract (BPE). In one embodiment, growth media comprising EGF and/or BPE are not filtered subsequent to the addition of EGF and/or BPE to the media. Additional growth media and conditions are described in the accompanying Examples.

Certain embodiments of the present in vitro methods are conducted in the form of high-throughput assays. As used herein, the term “high-throughput” means a process for assaying multiple samples for a desired biological activity or property. High-throughput assays are generally automated, and may allow for assaying multiple samples at the same time. In particular embodiments, the methods are performed using a plate with a plurality of wells or concavities, a plurality of which contain discrete cell populations, therefore allowing individual assays to be conducted. Plates used in high-throughput assays generally have 6, 12, 24, 48, 96, 384, or 1536 wells.

The use of a high-throughput platform allows for the standardization of assay conditions within a given set of experiments. High-throughput assays also reduce the experimental variation, both within and between experiments, as much of the assay can be automated. These features also lead to more meaningful comparisons of data among different trials. Other advantages of using in vitro high-throughput assays are that they generate far more data in a shorter time-frame. Moreover, performing the assays becomes are less labor intensive, more cost effective, and yield more reproducible results than in vitro assays conducted in a more piecemeal fashion.

It would be understood by one of ordinary skill in the art that any of the in vitro methods described herein are amenable to a high-throughput format, wherein the nephrotoxicity of a plurality of compounds or compound mixtures (and/or concentrations thereof) or the ability of a plurality of compounds or compound mixtures to act as nephroprotectants is assayed at a plurality of different concentrations.

### A. Determining Nephrotoxicity of a Compound

In one embodiment, the present invention provides a method of determining the nephrotoxicity of a test compound, comprising contacting kidney epithelial cells, e.g., HK-2 cells, with the test compound, and determining the level of an indicator of nephrotoxicity, wherein the level of the indicator is indicative of nephrotoxicity. In particular embodiments, the nephrotoxicity of the test compound is determined by comparing the level of the indicator to a control value. The control value may be a predetermined value based upon values obtained using one or more known nephrotoxic compounds, or it may be a control value determined using one or more known nephrotoxic compounds. Typically, at the same time, the test compound is being evaluated for nephrotoxicity.

In another embodiment, methods of the present invention measure the nephrotoxicity for a mixture of test compounds.

In a related embodiment, the methods of the present invention are used to determine or predict the in vivo nephrotoxicity of a test compound, by comparing the level of the indicator of nephrotoxicity produced by the test compound to the level(s) of the indicator of nephrotoxicity produced in the same in vitro assay by one or more known nephrotoxic compounds having a known in vivo nephrotoxicity. By comparing or correlating the level produced by the test compound to the level produced by one or more known nephrotoxic compounds, the in vivo nephrotoxicity of the test compound may be readily determined.

In particular embodiments, the levels of in vitro nephrotoxicity of a test compound are used to rank the nephrotoxicity of said test compound among one or more nephrotoxic compounds whose nephrotoxicity is known or that has been previously established by methods of the present invention.

A variety of different molecules may be employed as indicators of nephrotoxicity in the in vitro assays of the present invention. In particular embodiments, an indicator of nephrotoxicity is a measure of cell injury. In related embodiments damage to kidney cells is monitored by the release of brush border enzymes. In other embodiments, changes in kidney cell gene expression in genes such as osteopontin, inositol polyphospate multikinase, I-arginine glycine amidinotransferase, prosaposin, lipocalin, synaptogyrin 2, kallikrein, KIM-1, kidney injury molecule 1 (Kim1), lipocalin 2 (Lcn2) can be used to indicate nephrotoxicity of a compound. Examples of other genes that whose expression levels can be measure to indicate nephrotoxicity of a compound are describe in Amin et al., Environ Health Perspect. 2004 March; 112(4):465-79 and Wang et al., Toxicology. 2008 Jan. 16 [Epub ahead of print; PMID: 18289764], herein incorporated by reference in their entirety. One having ordinary skill in the art would appreciate that a variety of methods can be used to measure changes in gene expression, for example, DNA slot blot, RT-PCR, real-time PCR, oligonucleotide and cDNA microarrays, primer extension, S1 nuclease assay, and RNAse protection assays, among others.

In other embodiments of the present invention, the indicator of in vitro nephrotoxicity is a measure of cell death. The nephrotoxic effects of test compounds, and more particularly, aminoglycosides, may occur via several possible mechanisms, including inhibition of protein synthesis, mitochondrial injury, and DNA damage. These cellular insults ultimately lead to activation of programmed cell death pathways and apoptosis. Thus, indicators of in vitro nephrotoxicity suitable for use with the present invention are indicators of apoptosis. Indicators of apoptosis can be characterized by distinct morphologic changes consisting of cell shrinkage, nuclear condensation, and internucleosomal DNA fragmentation.

Preferably, in one embodiment, the in vitro indicator of nephrotoxicity is a biochemical indicator of apoptosis. For example, biochemical indicators of apoptosis may monitor caspase 8 activity, which is induced predominantly from apoptotic stimuli received via integral membrane death receptors such as Fas and TNFR1.

In another embodiment, biochemical indicators of apoptosis initiated from mitochondria may be assayed, such as caspase-9 activity or cytochrome C release. Alternatively, rather than monitoring apoptosis from a single pathway, a particular embodiment monitors the common effector of different apoptosis pathways, for example, caspase-3 activity. It has been established in the art that once activated, both caspases 8 and 9 participate in a cascade that culminates in the activation of caspase 3, which cleaves several substrates, resulting in chromosomal DNA fragmentation and cellular morphologic changes characteristic of apoptosis. Thus, it would be understood by one ordinarily skilled in the art that when the in vitro indicator of nephrotoxicity monitors caspase-3 activity, effectively all apoptotic pathway are being monitored.

In one embodiment, the indicator of in vitro nephrotoxicity is a morphological or biochemical indicator of apoptosis. In a particular embodiment, the indicator of in vitro nephrotoxicity is a biochemical marker of apoptosis such as cytochrome C oxidase activity, and caspase activity.

In a more particular embodiment, the caspase activity for use as an in vitro indicator of nephrotoxicity is selected from the group consisting of: caspase-9 activity, caspase-8 activity, caspase-7 activity, and caspase-3 activity. In another embodiment, the activities of one or more caspases selected from group consisting of caspase-9 activity, caspase-8 activity, caspase-7 activity, and caspase-3 activity are used as indicators of in vitro nephrotoxicity. In yet a more particular embodiment, the in vitro indicator of nephrotoxicity is caspase-3 activity.

In related embodiments, caspase activity is determined by using a conditionally activated luciferase substrate, wherein caspase cleavage of the luciferase substrate makes the substrate available to luciferase. In a particular embodiment, the luciferase substrate is specific for measuring caspase-3 activity. Such assays are provided by Promega under the name CASPASEGLO, which is a commercially available assay kit for monitoring caspase activity based upon luminescence.

The skilled artisan would appreciate that many other enzymes activated in apoptosis, and which are known in the art are equally suited for use as an in vitro indicator of nephrotoxicity in the in vitro nephrotoxicity assays described herein.

In various embodiments, the level of an in vitro indicator of nephrotoxicity is measured continuously. The levels of in vitro indicators of nephrotoxicity can be monitored at the time the compound or mixture of compounds are added to a culture of cells of the present invention. Monitoring can be done periodically, for example, at about 10 minutes, about 30 minutes, about 1 hour, about 2 hours, about 5 hours, about 12 hours, about 18 hours, about 24 hours, about 30 hours, about 36 hours, about 48 hours, about 56 hours, about 64 hours, about 72 hours or more after the compound or mixture of compounds are added to a culture of cells of the present invention. Monitoring can also be performed at a time between 0 hours and about 96 hours, 0 hours and about 84 hours, 0 hours and about 72 hours, 0 hours and about 60 hours, 0 hours and about 48 hours, 0 hours and about 36 hours, 0 hours and about 30 hours, or 0 hours and about 24 hours.

In particular embodiments, methods of the present invention comprise determining a dose response curve and/or an EC50 of a test compound or compound mixture. As used herein, the term “dose response curve” describes a relationship between the amount of a compound or mixture assayed and the resulting measured response. The term “dose” is commonly used to indicate the amount of the compound or mixture used in the experiment, while the term “response” refers to the measurable effect of the compound or compound mixture being tested. Dose-response relationships are determined graphically by plotting the varying compound or mixture concentration on the X-axis in log scale and the measurable response on the Y-axis. As used herein, the term “EC50” means the concentration of a compound or mixture of compounds that induces a response halfway between the baseline response and the maximum response of that compound or compound mixture.

In related embodiments, the nephrotoxicity of a test compound is determined by comparing the EC50 determined for a test compound by an in vitro assay of the present invention to the EC50 of one or more known nephrotoxic compounds, as determined using an in vitro assay of the present invention.

In other related embodiments, the nephrotoxicity of a test compound mixture is determined by comparing the EC50 determined for a test compound mixture by an in vitro assay of the present invention to the EC50 of one or more known nephrotoxic compounds or compound mixtures, as determined using an in vitro assay of the present invention. Generally, a dose-response curve for the test compound is determined by measuring the level of an indicator of nephrotoxicity produced using various concentrations of a test compound, such as a set of serial dilutions of the test compound. The goal of determining the nephrotoxicity of a test compound over a serially diluted concentration range is to provide for the construction of a dose response curve. The X-axis of a dose response curve generally represents the concentration of the test compound on a log scale, whereas the Y-axis represents the response of the in vitro indicator of nephrotoxicity to a particular concentration of test compound.

Those skilled in the art would understand that a standard dose-response curve is generally defined by four parameters: the baseline response, wherein the indicator of nephrotoxicity does not increase above the lowest concentration of test compound tested; the maximum response, wherein there is no additional increase in the in vitro indicator of nephrotoxicity with increasing concentrations of test compound; the slope of the curve, wherein changes in the in vitro indicator of nephrotoxicity increase with increasing test compound concentrations; and the EC50, wherein the concentration of test compound produces a half-maximal response in the in vitro indicator of nephrotoxicity for that given compound. More simply stated, the EC50 is the concentration of test compound that provokes a response half-way between the baseline response and maximum response.

Thus, the EC50 is a convenient measure of the inherent nephrotoxicity of a test compound. In addition, it would be understood by the skilled artisan that the relative nephrotoxicity of a test compound to a control nephrotoxic compound may be determined by comparing the EC50s of the test compound to the control nephrotoxic compound. Thus, a test compound is said to have a relatively high level of nephrotoxicity when the EC50 of the test compound is less than the EC50 of a control nephrotoxic compound. Likewise, the test compound is said to have a relatively low level of nephrotoxicity when the EC50 of the test compound is greater than the EC50 of a control nephrotoxic compound. The skilled artisan would understand that the methods of the present invention are able to determine all degrees of relative nephrotoxicity of a test compound to any given control nephrotoxic compound and that such methods are not limited to the examples above.

In particular embodiments of the present in vitro assays, a plurality of different concentrations of a test compound are assayed. This is useful in establishing a dose response curve and determining the EC50 for a test compound. In other embodiments, a plurality of different test compounds is assayed at essentially the same time. This is particularly useful in a screen to determine the nephrotoxicity of a large number of test compounds, e.g., to identify those compounds that are nephrotoxic and those compounds that are not nephrotoxic. In one particular embodiment, a plurality of test compounds are each assayed at a plurality of different concentration, e.g., in order to determine dose response curves and/or EC50 values for multiple test compounds.

In certain embodiments, the methods described herein measure the response of an in vitro indicator of nephrotoxicity to a plurality of test compound and control nephrotoxic compound concentrations in order to determine the nephrotoxicity of a test compound.

One aspect of the present invention provides methods that relate to measuring the nephrotoxicity of a compound using high-throughput in vitro assays. Nephrotoxicity determined by the in vitro methods described herein correlate well with in vivo indicators of nephrotoxicity, and thus, are reliable predictors of in vivo nephrotoxicity. In another aspect of the present invention, methods that measure the in vitro nephrotoxicity of a compound correlate well with the relative in vivo nephrotoxicity among different compounds assayed in a 14-day rat model of nephrotoxicity.

In one embodiment, a high-throughput method for determining the nephrotoxicity of a test compound comprises the steps of culturing cells in a plurality of wells of a tissue culture device; contacting the cells of each well with a particular concentration of test compound, selected from a plurality of test compound concentrations; determining an indicator of nephrotoxicity for each one of the plurality of different concentrations of the test compound in said contacted cells in order to produce a dose response curve; and determining the nephrotoxicity of said test compound, wherein the test compound may be any pharmaceutical compound.

In one embodiment, a high-throughput method for determining the nephrotoxicity of a test compound comprises the steps of culturing cells in a plurality of wells of a tissue culture device; contacting the cells of each well with a particular concentration of test compound or control nephrotoxic compound, selected from a plurality of test compound and control nephrotoxic compound concentrations; determining an indicator of nephrotoxicity for each one of the plurality of different concentrations of the test compound and the control nephrotoxic compounds in said contacted cells in order to produce a dose response curve of both the test compound and the control nephrotoxic compound; and determining the nephrotoxicity of said test compound relative to said control nephrotoxic compound.

In another embodiment, a high-throughput method for determining the nephrotoxicity of one or more test compounds comprises the steps of culturing cells in a plurality of wells of a tissue culture device; contacting the cells of each well with a particular concentration of a different test compound or control nephrotoxic compound(s), selected from a plurality of test compound and control nephrotoxic compound concentrations; determining an indicator of nephrotoxicity for each one of the plurality of different concentrations of each one or more test compound and control nephrotoxic compound(s) in said contacted cells in order to produce a dose response curve of both the one or more test compounds and the control nephrotoxic compound(s); and determining the nephrotoxicity of said one or more test compounds relative to said control nephrotoxic compound(s).

In another embodiment, the cells are cultured in a tissue culture device having a plurality of wells, wherein the plurality of wells is selected from the group consisting of 4, 6, 12, 24, 48, 96, 384, and 1536 wells. In another embodiment, the tissue culture device is a microtiter plate having a plurality of wells. In a particular embodiment, the microtiter plate may have 4, 6, 12, 24, 48, 96, 384, or 1536 wells. In a more particular embodiment, the microtiter plate may have 24, 48, 96, or 384 wells. In a preferred embodiment, the microtiter plate has 48, 96, or 384 wells. In another preferred embodiment, the microtiter plate has a plurality of wells selected from the group consisting of 24, 48, 96, and 384 wells.

In certain embodiments, cells of the present invention are seeded in 96 well plates in a volume of about 25 μl, 50 μl, 75 μl, 100 μl, 125 μl, 150 μl, 175 μl, or 200 μl. In particular embodiments, the density of seeded cells can be from about 100 cells/mL to 1,000,000 cells/mL, about 250 cells/mL to 100,000 cells/mL, about 500 cells/ml to 10,000 cells/mL, or about 1,000 cells/mL to 8,000 cells/mL. In related embodiments, the density of seeded cells is about 100 cell/mL, about 250 cell/mL, about 500 cell/mL, about 1000 cell/mL, about 2000 cell/mL, about 3000 cell/mL, about 4000 cell/mL, about 5000 cell/mL, about 6000 cell/mL, about 7000 cell/mL, about 8000 cell/mL, about 9000 cell/mL, about 10,000 cell/mL, about 50,000 cell/mL, about 100,000 cell/mL, about 500,000 cell/mL, or about 1,000,000 cell/mL, or any intervening density.

The present methods provide for testing the nephrotoxicity of a test compound. It would be understood by one of ordinary skill in the art that a test compound may be any pharmaceutical compound including small molecules and peptides that cause renal damage upon administration to a host. Such drugs include, by way of example, diuretics, NSAIDs, ACE inhibitors, cyclosporin, tacrolimus, radiocontrast media, interleukin-2, vasodilators (hydralazine, calcium-channel blockers, minoxidil, diazoxide), mitomycin C, conjugated estrogens, quinine, 5-fluorouracil, ticlopidine, clopidogrel, interferon, valacyclovir, gemcitabine, bleomycin, heparin, warfarin, streptokinase, nedaplatin, methoxyflurane, tetracycline, amphotericin B, cephaloridine, streptozocin, tacrolimus, carbamazepine, mithramycin, quinolones, foscamet, pentamidine, intravenous gammaglobulin, fosfamide, zoledronate, cidofovir, adefovir, tenofovir, mannitol, dextran, hydroxyethylstarch, lovastatin, ethanol, codeine, barbiturates, diazepam, quinine, quinidine, sulfonamides, hydralazine, triamterene, nitrofurantoin, mephenyloin, penicillin, methicillin ampicillin, rifampin, sulfonamides, thiazides, cimetidine, phenyloin, allopurinol, cephalosporins, cytosine arabinoside, furosemide, interferon, ciprofloxacin, clarithromycin, telithromycin, rofecoxib, pantoprazole, omeprazole, atazanavir, gold, penicillamine, captopril, lithium, mefenamate, fenoprofen, mercury, interferon, pamidronate, fenclofenac, tolmetin, foscamet, aciclovir, methotrexate, sulfanilamide, triamterene, indinavir, foscamet, ganciclovir, methysergide, ergotamine, dihydroergotamine, methyldopa, pindolol, hydralazine, atenolol, taxol, tumor necrosis factor, chlorambucil, interleukins, bleomycin, etoposide, fluorouracil, vinblastine, doxorubicin, cisplatin, aminoglycosides, and the like (see, generally, Devasmita et al., Nature Clinical Practice Nephrology (2006) 2, 80-91).

In one embodiment, the methods of the present invention determine the nephrotoxicity of compound that is classified as an aminoglycoside. In a particular embodiment, the compound assayed is an aminoglycoside antibiotic selected from the group consisting of gentamicins, kanamycins, streptomycins, amakicins, apramycins, netilmicins, paromomycins, tobramycins, and modified derivatives thereof.

In a more particular embodiment, the inherent nephrotoxicity is determined for a compound with either a known, suspected, or unknown level of nephrotoxicity.

The skilled artisan would understand that the methods of the present invention are particularly useful for establishing the nephrotoxicity of known, suspected, or unknown nephrotoxic compounds, because the in vitro methods described herein correlate well with in vivo indicators of nephrotoxicity, whereas previous measures of the nephrotoxicity in the art of such known, suspected, or unknown nephrotoxic compounds may not be truly indicative of their in vivo nephrotoxicity. The skilled artisan would also appreciate that in vitro assays of the present invention are suitable for establishing the rank order of in vivo nephrotoxicity among different known, suspected, or unknown nephrotoxic compounds.

In one embodiment, a high-throughput method for determining the nephrotoxicity of a test compound comprises the steps of culturing cells in a plurality of wells of a tissue culture device; contacting the cells of each well with a particular concentration of test compound, selected from a plurality of test compound concentrations; determining the levels of an indicator of nephrotoxicity for each one of the plurality of different concentrations of the test compound in said contacted cells in order to produce a dose response curve; and determining the nephrotoxicity of said test compound, wherein the plurality of concentrations of the test compound is a serially diluted range of concentrations within the range of about 0 μg/mL to about 1 mg/mL.

In one embodiment, in vitro nephrotoxicity is assayed for a plurality of different concentrations of the test compound or compound mixture with the goal of including some concentrations at which no toxic effect is observed and also at least two or more higher concentrations at which a toxic effect is observed. For example, assaying test compounds at several concentrations within the range of about 0 μg/mL to about 1 mg/mL is commonly useful to achieve these goals. It will be possible or even desirable to conduct certain of these assays at concentrations of about 0.05 μg/mL to about 750 μg/mL, or of about 0.5 μg/mL to about 500 μg/mL. In a particular embodiment, the range of concentrations to be tested consists of a plurality of 2, 3, 4, 5, 6, 7, 8, 9, or 10 or more different concentrations within a range of about 0 μg/mL to about 1 mg/mL, or alternatively, within a concentration range of about 0.5 μg/mL to about 500 μg/mL.

In a more particular embodiment, the plurality of different test compounds or combinations of compounds are 2-fold serial dilutions, 3-fold serial dilutions, 4-fold serial dilutions, 5-fold serial dilutions, 6-fold serial dilutions, 7-fold serial dilutions, 8-fold serial dilutions, 9-fold serial dilutions, or 10-fold serial dilutions in a range of concentrations of about 0 μg/mL to about 1 mg/mL, or alternatively, within a concentration range of about 0.5 μg/mL to about 500 μg/mL. For example, a series of ten, 2-fold serial dilutions of a test compound starting at a concentration of about 500 μg/mL would include test compound concentrations of about 500 μg/mL, about 250 μg/mL, about 125 μg/mL, about 62.5 μg/mL, about 31.3 μg/mL, about 15.6 μg/mL, about 7.8 μg/mL, about 3.9 μg/mL, about 2 μg/mL, about 1 μg/mL, and about 0.5 μg/mL.

It would be understood by those skilled in the art that the particular concentrations assayed in an in vitro nephrotoxicity assay of the present invention may vary from one test compound to another. Moreover, the concentrations assayed for a mixture of compounds varies depending on the compounds in the mixture, and thus, mixtures of compounds may contain lower, the same as, or greater concentrations of compounds than would be present in an individual nephrotoxicity assay of a compound in the mixture.

In one embodiment, the dose response curve of one or more test compounds is compared to the dose response curves for one or more control nephrotoxic compounds. In certain embodiments, the control nephrotoxic compounds can be aminoglycosides, wherein the nephrotoxicity of said control nephrotoxic compound is known. In a related embodiment the one or more control nephrotoxic compounds can be aminoglycoside antibiotics. In another related embodiment one or more control nephrotoxic compounds are aminoglycoside antibiotics selected from the group of gentamicins, kanamycins, streptomycins, amakicins, apramycins, netilmicins, paromomycins, tobramycins, and modified derivatives thereof, wherein the nephrotoxicity is known. In another embodiment, the one or more control nephrotoxic compounds are aminoglycoside antibiotics selected from the group of gentamicins, kanamycins, amakicins, and apramycins. In a particular embodiment, the one or more control nephrotoxic compounds are the aminoglycoside antibiotics gentamicin and amikacin.

It would be readily understood to those of skill in the art that once a dose response curve is known for a particular test compound, that test compound is capable of being utilized as a control nephrotoxic compound. The skilled artisan would also recognize that the control nephrotoxic compounds and test compounds should not be the same compound in a given assay.

In a related embodiment, the relative nephrotoxicity of a test compound or compound mixture is determined by comparing the dose response curve for the test compound or mixture to the dose response curve for one or more control nephrotoxic compounds.

An important feature of the novel methods of the present invention described herein is the extremely high correlation between these in vitro nephrotoxicity assay and the in vivo indicators of nephrotoxicity such as blood, urea, nitrogen levels (BUN), serum creatinine levels, and histopathological indicators of nephrotoxicity.

In particular embodiments, the in vitro indicator of nephrotoxicity that measures apoptosis correlates well with one or more in vivo indicators of nephrotoxicity, e.g., an in vivo indicator of nephrotoxicity selected from the group consisting of BUN, serum creatinine, and histopathology.

In more particular embodiments, the in vitro indicator of nephrotoxicity measures caspase activity, which correlates well with the minimum concentration of compound required in vivo to cause increases in BUN and serum creatinine measured in a nephrotoxicity animal model. In specific embodiments the indicator of in vitro nephrotoxicity is caspase-3 activity, measured by luciferase, which correlates well with in vivo increase in BUN levels in a 14 day nephrotoxicity rat model.

The skilled artisan would readily understand that “correlates well” is a relative term and that many methods are known in the art to calculate correlations. Furthermore, the skilled artisan is knowledgeable regarding what constitutes a significant correlation and may make that determination without further guidance provided herein. An in vitro EC50 of a test compound that correlates well with the minimum concentration of test compound in vivo to cause an increase in an indicator of nephrotoxicity can have a correlation of about 0.75 to about 1, about 0.8 to about 1, about 0.85 to about 1, about 0.9 to about 1, about 0.95 to about 1 or any numerical value between 0.75 and 1.

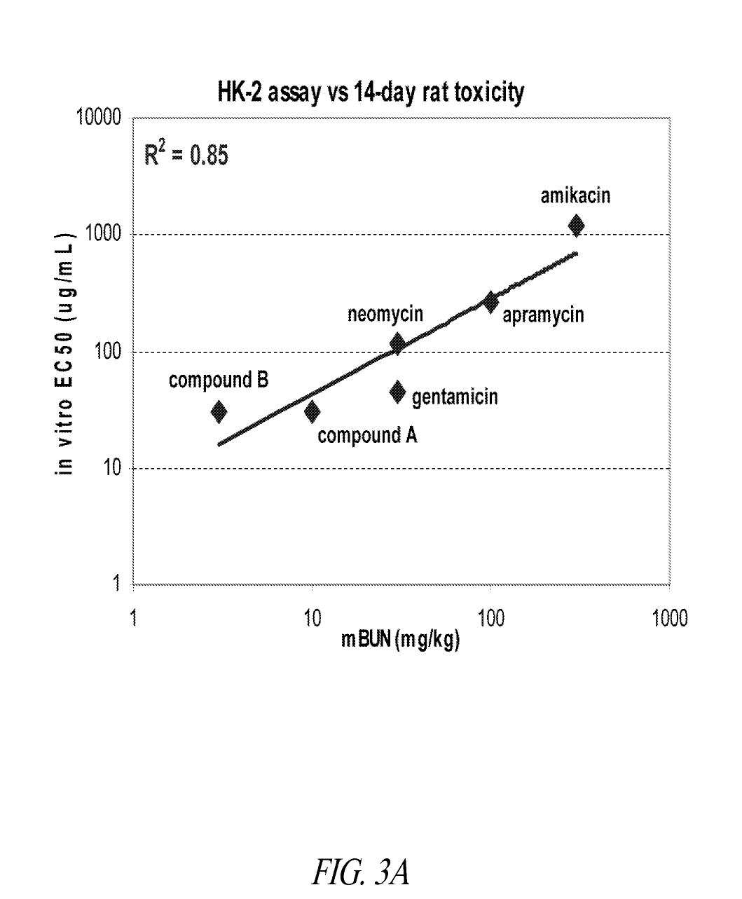

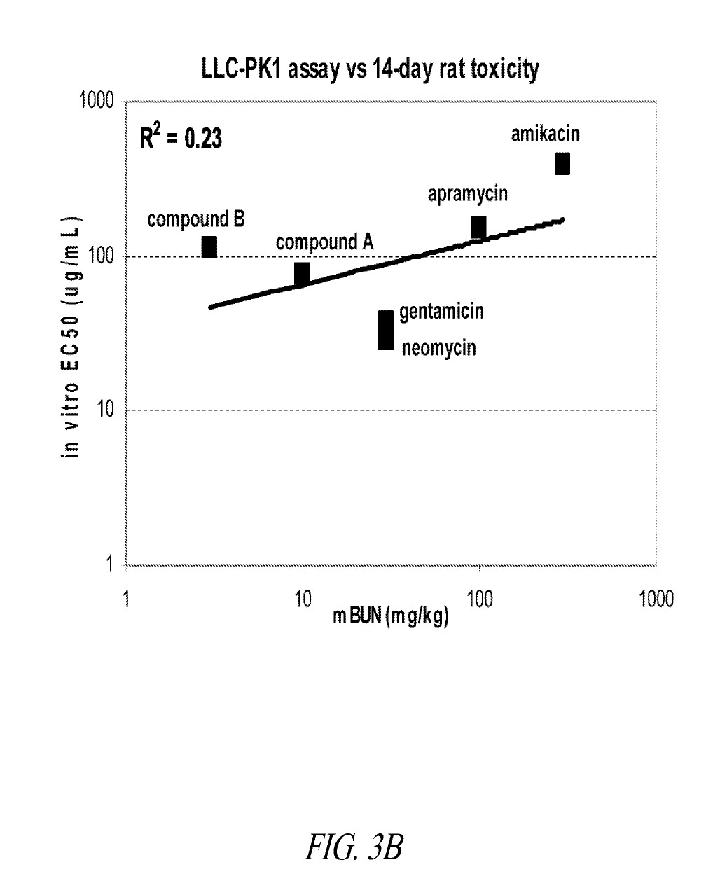

For instance, as shown in FIG. 3, a nephrotoxicity assay performed in HK-2 cells of control nephrotoxic compounds, gentamicin, amikacin, apramycin, neomycin, and test compounds A and B correlates well with the minimum concentrations of these compounds in vivo that cause increases in BUN (FIG. 3A). In contrast, when the in vitro assay is carried out in LLC-PK1 cells, gentamicin, amikacin, apramycin, neomycin, and test compounds A and B fail to correlate with minimum concentrations of these compounds in vivo that cause increases in BUN (FIG. 3B). However, as FIG. 4 clearly demonstrates, the in vitro nephrotoxicity measured for a group of compounds in an LLC-PK1 assay correlates well with the relative in vivo nephrotoxicity of these compounds in a 14-day rat nephrotoxicity model.

### B. Identification of Nephroprotectant Compounds

The methods described above may be readily adapted for the identification of nephroprotectant compounds, including those compounds that protect against specific nephrotoxic compounds or classes thereof, and those that protect against multiple or all nephrotoxic compounds. The art has failed to identify a sufficient number of useful nephroprotectant compounds for human use. Moreover, there is currently insufficient data regarding the ability of a single nephroprotectant to protect against a plurality of nephrotoxic compounds.

In one embodiment, the present invention provides an assay to determine the ability of a compound or combination of compounds to act as a nephroprotectant, comprising contacting human kidney epithelial cells, e.g., HK-2 cells, with a candidate nephroprotectant and a nephrotoxic compound, and determining a level of nephrotoxicity as described above, wherein a decrease in the level of nephrotoxicity indicates the candidate nephroprotectant acts as a nephroprotectant against the nephrotoxic compound. In another embodiment, one or more nephroprotectants are assayed in parallel against a particular nephrotoxic compound or a group of nephrotoxic compounds.

In particular embodiments, the present invention provides an assay to determine the ability of a compound to act as a nephroprotectant, comprising contact porcine kidney tubule cells, e.g., LLC-PK1 cells, with a candidate nephroprotectant and a nephrotoxic compound, and determining a level of nephrotoxicity as described above, wherein a decrease in the level of nephrotoxicity indicates the candidate nephroprotectant acts as a nephroprotectant against the nephrotoxic compound.

Certain embodiments of the present invention provide for large scale high-throughput screening of small molecule libraries to identify compounds with the ability to provide a nephroprotective effect. Some advantages in using the high-throughput screens of the present invention is that more nephroprotectant compounds can be identified, and the nephroprotective effects of these compounds can be directly compared in a single assay. Additionally, in certain embodiments, broad spectrum nephroprotective compounds or mixtures can be identified by assaying the nephroprotective effects of a compound against a variety of nephrotoxic compounds.

In certain embodiments, cultured cells described elsewhere herein are suitable for use with the present invention of determining the ability of a compound to act as a nephroprotectant. For example, cells may be primary kidney epithelial cell cultures or cultured kidney epithelial cell lines. In particular embodiments, primate epithelial kidney cells are well suited for the screening of nephroprotectant compounds. In a specific embodiment, a human epithelial kidney cell line such as HK-2 cells is the preferred cells to use in nephroprotectant screens. In other embodiments, LLC-PK1 cells are used in nephroprotectant screens.

In order to be characterized as a nephroprotectant compound, increasing concentrations of the candidate nephroprotectant compound or combination of compounds should elicit a dose dependent decrease in the levels of the in vitro indicator of nephrotoxicity in the presence and/or absence of the nephrotoxic compound.

In one embodiment screening for the ability of one or more candidate nephroprotectant compounds or mixtures to act as a nephroprotectant, comprises the steps of: (i) contacting discrete populations of HK-2 cells with a plurality of different concentrations of said one or more candidate nephroprotectant compounds, wherein each of a plurality of different concentrations for each of said one or more test compounds contacts a separate discrete population of HK-2 cells; (ii) contacting additional discrete populations of HK-2 cells with a plurality of different concentrations of said one or more candidate nephroprotectant compounds as in step (i), and further contacting all the additional discrete populations of HK-2 cells with a static concentration of nephrotoxic compound; (iii) determining the levels of an indicator of nephrotoxicity for each one of the contacted populations of HK-2 cells of step (i) and step (ii); and (iv) determining the ability of each one or more candidate nephroprotectant compounds to act as a nephroprotectant.

In further embodiments, an in vitro indicator of nephrotoxicity for a nephrotoxic compound or compound mixture exhibits a dose dependent signal reduction when exposed to increasing concentrations of candidate nephroprotectant compound. In a related embodiment, the level of apoptosis activity elicited by a static concentration of nephrotoxic compound such as an aminoglycoside exhibits a dose dependent signal decrease in response to an increasing concentration of candidate nephroprotectant compound. In another related embodiment, the level of caspase activity for a aminoglycoside antibiotic selected from the group consisting of gentamicins, kanamycins, streptomycins, amakicins, apramycins, netilmicins, paromomycins, tobramycins, and modified derivatives thereof, exhibits a dose dependent signal decrease in response to an increasing concentration of candidate nephroprotectant compound.

In a specific embodiment, the level of caspase-3 activity for a control aminoglycoside antibiotic such as gentamicin exhibits a dose dependent signal decrease in response to an increasing concentration of candidate nephroprotectant compound or mixture.

The skilled artisan would appreciate that when the candidate nephroprotectant compound specifically reduces the apoptosis signal caused by a nephrotoxic compound in a dose-dependent fashion, but without altering the background apoptosis signal present in the absence of a nephrotoxic compound, the nephroprotectant is specific nephrotoxic compounds. In addition, if a candidate nephroprotectant compound causes a dose dependent decrease in apoptosis signal elicited by one specific nephrotoxic compound, (e.g., gentamicin), then the nephroprotectant is specific for that nephrotoxic compound.

Alternatively, in a related embodiment, the candidate nephroprotectant compound may reduce both the background levels of apoptosis in the cultured cells lacking a nephrotoxic compound and the apoptosis induced by the control nephrotoxic agent. This type of result suggests that the candidate nephroprotectant compound acts on the particular control nephrotoxic compound, as well as at a different point in the apoptosis pathway.

In a particular embodiment, when the control nephrotoxic compound is an aminoglycoside antibiotic, this dual mode of action by a given candidate nephroprotectant compound should be relevant to enhancing the clinical utility of aminoglycosides, since both the specific aminoglycoside and the apoptosis response are blocked.

The skilled artisan would understand that many compounds may possess biochemical activities sufficient to render them useful as nephroprotectants, and thus, the present invention provides for screening a wide range of candidate nephroprotectant compounds. As used herein, a candidate nephroprotectant compound can be any chemical compound, for example, a macromolecule (e.g., a polypeptide, a protein complex, glycoprotein, or a nucleic acid) or a small molecule (e.g., an amino acid, a nucleotide, an organic or inorganic compound).

A candidate nephroprotectant compound can have a formula weight of less than about 10,000 grams per mole, less than 5,000 grams per mole, less than 1,000 grams per mole, or less than about 500 grams per mole.

The candidate nephroprotectant compound can be naturally occurring (e.g., an herb or a natural product), synthetic, or can include both natural and synthetic components. Examples of candidate nephroprotectant compounds include antioxidants, compounds that structurally resemble antioxidants, peptides, peptidomimetics (e.g., peptoids), amino acids, amino acid analogs, poly-aspartic acid, polynucleotides, polynucleotide analogs, polyphenolic acids, nucleotides, nucleotide analogs, and organic (e.g., aspirin, and benzoic acid derivatives) or inorganic compounds (e.g., organometallic compounds).

In particular embodiments, combinations or mixtures of the above mentioned classes of nephroprotectant compounds are screened.

In certain embodiments the nephroprotectant compound screening assay may additionally comprise the use of a control nephroprotectant compound. It would be understood by one of skill in the art that once the nephroprotective ability of a compound is known, that compound may be suitable for use as a control nephroprotectant compound in the in vitro assays of the present invention.

In another embodiment, the ability of a compound to act as a broad spectrum nephroprotectant compound is assayed. In order to identify a broad spectrum nephroprotectant compound, the ability of the nephroprotectant compound would be assayed in the presence and absence of 1 or more, 2 or more, 3 or more, 4 or more, 5 or more, 6 or more, 7 or more, 8 or more, 9 or more, or 10 or more nephrotoxic compounds.

For example, a particular candidate nephrotoxic compound can be screened against various aminoglycoside antibiotics. In a particular embodiment, the candidate nephroprotectant is screened for nephroprotective ability against the aminoglycoside antibiotics selected from the group consisting of gentamicins, kanamycins, streptomycins, amakicins, apramycins, netilmicins, paromomycins, tobramycins, and modified derivatives thereof, wherein the nephrotoxicity is known. In another embodiment, the nephrotoxic compounds are aminoglycoside antibiotics selected from the group of gentamicins, kanamycins, amakicins, and apramycins. In a particular embodiment, the nephrotoxic compounds are the aminoglycoside antibiotics gentamicin and amikacin.

The skilled artisan would appreciate that the more than one candidate or combination of candidates can be screened at the same time against a variety of nephrotoxic compounds as described above herein.

In one embodiment, libraries of small molecules can be screened for the ability to act as a nephroprotectant against one or more nephrotoxic compounds. A high-throughput screen for candidate nephroprotectant compounds comprises the steps of culturing cells in a plurality of wells of a tissue culture device; contacting the cells of each well with a particular concentration of candidate nephroprotectant compound with or without a static concentration of nephrotoxic compound, wherein the candidate nephroprotectant compound concentration is selected from a plurality different concentrations; detecting the levels of an indicator of nephrotoxicity for each one of the plurality of different concentrations of the candidate nephroprotectant compound in said contacted cells either in the presence or absence of the static concentration of nephrotoxic compound in order to identify the ability of the candidate nephroprotectant compound to act as a nephroprotectant against said nephrotoxic compound.

In related embodiments, a high-throughput screen for candidate nephroprotectant compounds comprises the steps of culturing cells in a plurality of wells of a tissue culture device; contacting the cells of each well with a particular concentration of candidate nephroprotectant compound with or without a static concentration of nephrotoxic compound, wherein the candidate nephroprotectant compound concentration is selected from a plurality different concentrations; monitoring an indicator of nephrotoxicity for each one of the plurality of different concentrations of the candidate nephroprotectant compound in said contacted cells either in the presence or absence of the static concentration of nephrotoxic compound in order to identify the ability of the candidate nephroprotectant compound to act as a nephroprotectant against said nephrotoxic compound, wherein the highest concentration of candidate nephroprotectant compound tested is greater than the static concentration of nephrotoxic compound.

In related embodiments, the candidate nephroprotectant compound is assayed at a plurality of concentrations.

The highest concentration of candidate nephroprotectant compound tested is about at least 50, about at least 40, about at least 30, about at least 20 or about at least 10 times the concentration of control nephrotoxic compound used in the assay.

The control nephrotoxic can be any compound wherein the dose response curve for nephrotoxicity is known. The concentration of control nephrotoxic compound used in the candidate nephroprotectant compound assay is the concentration where there is about 90%, about 95%, 96%, 97%, 98%, 99%, or 100% nephrotoxicity, or alternatively, the concentration necessary to initiate a uniform apoptosis response.

In one embodiment, the number of candidate nephroprotectant compound concentrations assayed is about 20, about 15, about 10, about 5, or about 1, or any integer value between about 1 and about 20.

In a more particular embodiment, gentamicin is the control nephrotoxic compound and is added to all assays at a concentration of 100 ug/mL.

In a related embodiment, the candidate nephroprotectant compound is added at a concentration range of about 5000 ug/mL to about 0 ug/mL, about 2500 ug/mL to about 1 ug/mL, or about 2000 ug/mL to about 2 ug/mL.

In another related embodiment, the plurality of different candidate nephroprotectant compound concentrations are 2-fold serial dilutions, 3-fold serial dilutions, 4-fold serial dilutions, 5-fold serial dilutions, 6-fold serial dilutions, 7-fold serial dilutions, 8-fold serial dilutions, 9-fold serial dilutions, or 10-fold serial dilutions in a range of concentrations of about 2 ug/mL to about 2000 ug/mL.

In various related embodiments, the level of an in vitro indicator of nephrotoxicity measured in a nephroprotectant assay of the present invention is measured continuously. In related embodiments, the ability of a compound to act as a nephroprotectant can be monitored at the time the nephroprotectant compound or mixture of compounds are added to a culture of cells containing an aminoglycoside antibiotic. In particular embodiments, monitoring can be done periodically, for example, at about 10 minutes, about 30 minutes, about 1 hour, about 2 hours, about 5 hours, about 12 hours, about 18 hours, about 24 hours, about 30 hours, about 36 hours, about 48 hours, about 56 hours, about 64 hours, about 72 hours or more after the nephroprotectant compound or mixture of compounds are added to a culture of cells containing an aminoglycoside antibiotic. In related embodiments, monitoring can also be performed at a time between 0 hours and about 96 hours, 0 hours and about 84 hours, 0 hours and about 72 hours, 0 hours and about 60 hours, 0 hours and about 48 hours, 0 hours and about 36 hours, 0 hours and about 30 hours, or 0 hours and about 24 hours.

The methods of the present invention also provide for nephroprotectant validation assays or counterscreens. As used herein, the term “counterscreen” means an assay that is used subsequent to an initial screening assay to identify nephroprotectants. Counterscreens are employed in order to validate and further describe the properties of candidate molecules identified in screens as true positives. In some instances, counterscreens are used to eliminate molecules identified in a screening assay that possess undesired activities. Candidate nephroprotectant compounds from an initial screen are subjected to a series of counterscreens to discard any that interfere with the assay readout rather than inhibit nephrotoxicity induced by a nephrotoxic compound, such as an aminoglycoside (e.g., gentamicin).

One explanation for the source of a false positive result in an initial nephroprotectant screen is a reduction in apoptosis (e.g., caspase activity) signal caused by the fact that the candidate nephroprotectant itself is toxic to the cultured cells.

In one embodiment, a cell viability assay is used to rule out this class of false positive, wherein the same concentrations of candidate nephroprotectant compound are used in the presence of a viability marker. Various viability markers may be used to determine cell viability. For example, vital dyes such as azafloxin, basic blue (nile blue sulphate), bismarck brown, basic red (rhodamine 6G), bengal red, brilliant crysyl blue, eosin, fluorescein, gentian violet, indocyanine green, janus green, methylene green, methylene blue, neutral red, trypan blue, trypan red, and tetrazolium salts may be used in the methods of the present invention to determine cell viability.

Alternatively, two other sources of false positives may be linked to a particular embodiment of the present invention, wherein the in vitro indicator of nephrotoxicity is a luciferase substrate that is conditionally activated by caspase activity (i.e., a caspase/luciferase substrate such as the CASPASEGLO reporter molecule). For example, in the in vitro assays described herein, luminescence from a CASPASEGLO substrate is only detected in the presence of both luciferase and caspase enzymatic activities, and thus, a candidate nephroprotectant compound that targets either luciferase or caspases would yield a false positive result.

Although compounds that inhibit luciferase are rare, a second counterscreen can be employed to eliminate this type of false positive. In one embodiment luciferase activity is conditionally active in viable cells, and thus, if the candidate nephroprotectant compound causes a dose-dependent decrease in this assay, the candidate nephroprotectant compound is likely to be specific for luciferase and can reliably be detected as a false positive. For example, the fact that luciferase requires ATP to produce bioluminescence from a luciferin substrate makes for a luminescence measure that is conditionally active in viable cells, as only viable cells generate ATP. Commercial kits to accomplish this type of assay are available from Promega (e.g., CELLTITERGLO). In a particular embodiment, the inhibition ATP dependent bioluminescence elicited by a candidate nephroprotectant compound would suggest the candidate nephroprotectant compound is not specific for a given nephrotoxic compound but rather directly inhibits luciferase activity.

Another source of false positives, as mentioned above, would be candidate nephroprotectant compounds that directly inhibit caspase activity. In one embodiment, an in vitro counterscreen is employed that determines the ability of the candidate nephroprotectant to inhibit the cleavage of a caspase substrate. Perhaps the most convenient way to measure caspase activity is by using conditionally active fluorogenic caspase substrates. These molecules may comprise a fluorogenic molecule such as aminomethylcoumarin (AMC), aminotrifluoromethylcoumarin (AFC), rhodamine 110, GFP, and the like, and a caspase specific peptide, that when cleaved will allow the fluorophore to become active. Many examples of caspase specific peptides are known in the art. For example: YVAD is a peptide specific for caspases 1 and 4; VDVAD is a peptide specific for caspase 2; and DEVD is a peptide specific for caspases 3, 6, 7, 8, and 10. However, candidate nephroprotective molecules that inhibit caspases need not be discarded, as caspase inhibitors would still act as nephroprotectants to block nephrotoxicity, which is known in the art to be mediated by apoptosis.

The in vitro assays of the present invention also provide reagents that can comprise part of a kit. Such kits are useful in standardizing assays to increase the reliability of results. Kits also present an opportunity to decrease costs and increase the ease of manipulation associated with the assays of the present invention.

In one embodiment, a kit is supplied for determining the nephrotoxicity of a compound. Such kits comprise instructions for using the kit, which are optionally accompanied by a table of previously established dose response curves and EC50s of known nephrotoxic compounds to facilitate the determination of nephrotoxicity of a compound. These kits further comprise a multi-well culture vessel of about 24, 48, 96, 384 or 1536 wells. Such culture vessels can be microtiter plates, tissue culture plates, slides, and the like. Kits also include an in vitro indicator of nephrotoxicity, such as a luciferase substrate specific for caspase activity and those described elsewhere herein. Kits may further comprise one or more control nephrotoxic compounds. Kits optionally comprise an epithelial kidney cell line, optionally the human epithelial kidney cell line HK-2.

In another embodiment, a kit is supplied for determining the ability of a candidate nephroprotectant compound to act as a nephroprotectant. Such kits comprise instructions for using the kit; a multi-well culture vessel of about 24, 48, 96, 384 or 1536 wells; a luciferase substrate specific for caspase activity; a control nephrotoxic compound; and reagents to be used in counterscreens. Counterscreening agents comprised in the kit are cell viability determining reagents (e.g., vital dyes) and luciferase assays conditional on cell viability, such as those described herein. Optionally, kits may comprise a variety of well known aminoglycosides, such as those discussed herein, to use in screening for effective nephroprotectants. Kits optionally comprise an epithelial kidney cell line, optionally the human epithelial kidney cell line HK-2.

In another embodiment, kits of the present invention comprise the LLC-PK1 cell line or porcine kidney tubule cells.

While the terms used in the application are intended to be interpreted with the ordinary meaning as understood by persons skilled in the art, some terms are expressly defined in order to avoid ambiguity.

Prior to explaining at least one embodiment of the invention in detail by way of exemplary figures, experimentation, results, and laboratory procedures, it is to be understood that the invention is not limited in its application to the details of construction and the arrangement of the components set forth in the following description or illustrated in the figures, experimentation and/or results. The invention is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

### EXAMPLES

### Example 1

### HK-2 Nephrotoxicity Assay

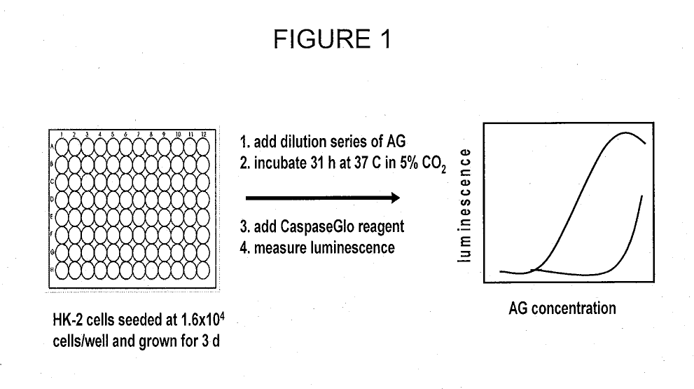

An in vitro nephrotoxicity assay was conducted using the human kidney epithelial cell line, HK-2. An overview of the HK-2 nephrotoxicity assay protocol is shown in FIG. 1.

HK-2 cells were cultured in KSFM, with 5 ng/mL epidermal growth factor (EGF) and 0.05 mg/mL bovine pituitary extract (BPE). Cells were maintained at sub-confluence and used to initiate the assay when they reached 80% confluence. HK-2 cells were harvested using trypsin-EDTA and dispensed into the wells of a 96-well polystyrene tissue-culture plate at a density of 1.6×10⁴ cells/well in a final volume of 100 uL/well. Plates were incubated at 37° C. with 5% CO₂ for 3 days.

Compounds to be tested were diluted in KSFM with 10 mM HEPES buffer that had been pre-warmed to 37° C. Plates were removed from the incubator and the media was removed using a multichannel aspirator. Diluted compounds were added to plates. The plates were subsequently returned to the incubator overnight.

Plates were removed from the incubator and allowed to cool to room temperature. 50 uL of CASPASEGLO (Promega) was added to each well. The plates were briefly agitated on a shaker platform and then allowed to sit at room temperature for 30 minutes. Luminescence was detected using a plate reading luminometer. Increases in luminescence over the background signal from untreated cells was indicative of apoptosis induced by the addition of compound.

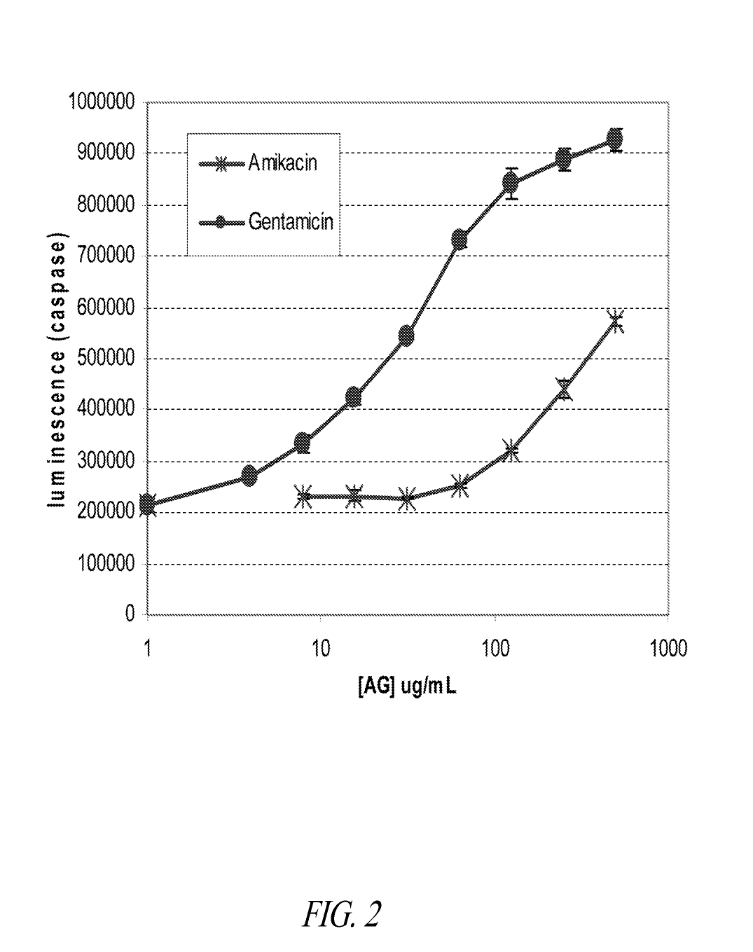

Novel compounds were usually tested in parallel with amikacin and gentamicin controls on each plate. Typical control data is shown in FIG. 2. Gentamicin produced a full dose response curve over the concentration range tested and an EC50 was calculated. As the concentration of aminoglycoside increased, the luminescence signal corresponding to induction of apoptosis also increased. Amikacin did not produce a maximal response at the highest concentration tested (500 μg/mL), and therefore, the EC50 could only be estimated.

In a pilot study, it was found that a majority of the novel compounds screened had dose response curves distributed between gentamicin and amikacin, such that EC50s could be calculated. The apoptosis signal for some of the compounds screened was non-zero in the absence of aminoglycoside. This background signal fluctuated with the number of cells and was likely indicative of background levels of apoptosis taking place in the cell culture.

In another study, more than 400 novel aminoglycosides were screened in a high-throughput format for in vitro nephrotoxicity in HK-2 cells using the methods described herein. Utilization of the high-throughput format required orders of magnitude less compound (240 μg) than the corresponding standard 14-day rat nephrotoxicity assay (6 g).

### Example 2

### Correlation of In Vitro HK-2 Assay with In Vivo Nephrotoxicity

To validate the utility of the HK-2 assay described in Example 1, the nephrotoxicity of four commercial aminoglycoside antibiotics (amikacin, gentamicin, neomycin and apramycin) and two other test compounds (compounds A and B) were tested using both the in vitro HK-2 assay and a 14-day rat nephrotoxicity model. The rat in vivo assays provided information in the form of acute toxicity, changes in Blood Urea Nitrogen (BUN) and serum creatinine levels over time, as well as histopathology changes in the kidneys of animals sacrificed at the end of the study. Elevation in BUN or serum creatinine is one of the markers routinely monitored in the clinic as an indication of aminoglycoside nephrotoxicity.

These studies demonstrated a significant correlation between the results from the in vitro HK-2 assay and the in vivo results obtained from the 14-day rat nephrotoxicity model. Specifically, it was found that the minimum concentration of compound that produced an increase in BUN in the in vivo rat model correlated well with the EC50 from the in vitro HK-2 assay (see FIG. 3A). Both assays indicated that amikacin was the least nephrotoxic aminoglycoside tested, followed by apramycin, gentamicin and neomycin, compound A, and compound B. The results obtained with these six compounds suggested that both the in vitro HK-2 assay and the in vivo rat model assay ranked the toxicity of the compounds in the same order, and therefore, the in vitro HK-2 assay served as a suitable predictor of in vivo nephrotoxicity established in the 14-day rat study.

In direct contrast, the correlation between the in vitro LLC-PK1 assay and the in vivo rat model was poor (FIG. 3B), and demonstrated that the LLC-PK1 assay was not suitable to predict in vivo nephrotoxicity. These results demonstrate that HK-2 cells represent a particularly useful nephrotoxicity assay. Moreover, the results obtained using these particular cells correlates well with in vivo nephrotoxicity.

### Example 3

### Correlation of In Vitro LLC-PK1 Assay with In Vivo Relative Nephrotoxicity

An in vitro nephrotoxicity assay was conducted using the porcine kidney tubule cell line, LLC-PK1. Assays are conducted essentially as described in Example 6, but with the modifications.

On Day 1 of the experiment, LLC-PK1 cells that had been maintained in low glucose DMEM with 5% FBS were trypsinized and re-seeded at a density of 8×10³ cell/well in the wells of a 96-well polystyrene tissue-culture plate. Plates were incubated at 37° C. with 5% CO₂ for 3 days.

On day 4, compounds to be tested were diluted in Ultraculture medium (Cambrex) containing 2 mM L-glutamine and 20 mM HEPES buffer that had been pre-warmed to 37° C. The compounds that were tested were serially diluted from 400 μg/mL to 1 μg/mL. Plates were removed from the incubator and the media was removed using a multichannel aspirator. Cells were washed with Ultraculture medium and then the diluted compounds were added to plates. The plates were subsequently returned to the incubator overnight.

On day 5, plates were removed from the incubator and allowed to cool to room temperature. 50 uL of CASPASEGLO (Promega) was added to each well. The plates were briefly agitated on a shaker platform and then allowed to sit at room temperature for 30 minutes. Luminescence was detected using a plate reading luminometer. Increases in luminescence over the background signal from untreated cells was indicative of apoptosis induced by the addition of compound. Novel compounds were usually tested in parallel with amikacin and gentamicin controls on each plate. FIG. 4 shows the data generated from an in vitro LLC-PK1 experiment performed with Gentamicin, Kanamycin, Amikacin, and Streptomycin correlated well with the relative nephrotoxicity of these aminoglycosides in an in vivo 14-day rat nephrotoxicity study.

This study demonstrated a significant correlation between the results from the in vitro LLC-PK1 assay and the relative in vivo nephrotoxicity obtained from a 14-day rat nephrotoxicity model. Specifically, it was found that the minimum concentration of compound that produced an increase apoptosis in the in vivo rat model correlated well with the levels of apoptosis measured in the in vitro LLC-PK1 assay (see FIG. 4). Both assays indicated that streptomycin was the least nephrotoxic aminoglycoside tested, followed by amikacin, neomycin, and gentamicin. The results obtained with these four compounds suggested that both the in vitro LLC-PK1 assay and the in vivo 14-day rat nephrotoxicity model assay ranked the toxicity of the compounds in the same order, and therefore, the in vitro LLC-PK1 assay served as a suitable predictor of the relative in vivo nephrotoxicity of these compounds.

### Example 4

### Identification of Nephroprotectants

The nephrotoxicity assay was adapted to perform screens to identify nephroprotectants. HK-2 cells were cultured as in Example 1. In a typical example of a nephroprotectant screen, gentamicin was added to all wells at 100 ug/mL to initiate a uniform apoptosis response. At the same time, a dilution series of candidate nephroprotectants was added. The highest nephroprotectant concentration tested was 20 times the gentamicin concentration present in the assay. A dose-dependent reduction in the gentamicin-induced apoptosis signal was suggestive of an additive that is serving to block nephrotoxicity in those cells.

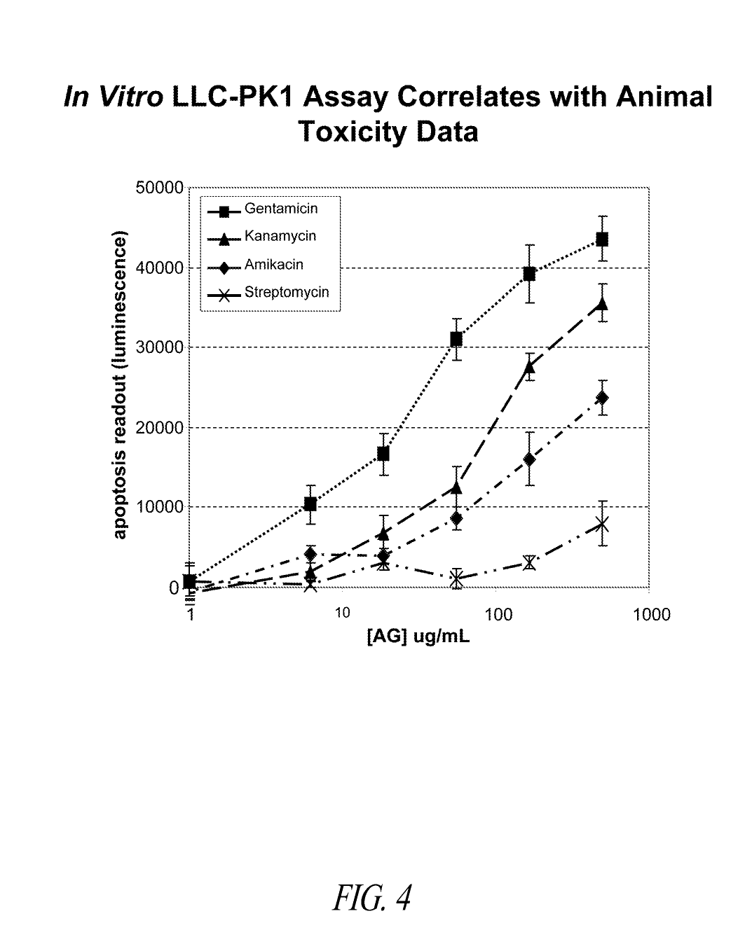

FIG. 5 shows an example of a potential nephroprotectant screened in the in vitro assay. During screening both aminoglycoside-specific and non-specific nephroprotectants was observed. The aminoglycoside-specific compounds reduced the gentamicin induced apoptosis signal without altering the background apoptosis signal. The second class of putative nephroprotectants caused a reduction in both the gentamicin induced and background apoptosis signals. This demonstrated that the two groups of molecules acted at different points in the apoptosis response, both of which are relevant to enhancing the clinical utility of aminoglycosides.

### Example 5

### Counterscreens of Candidate Nephroprotectants

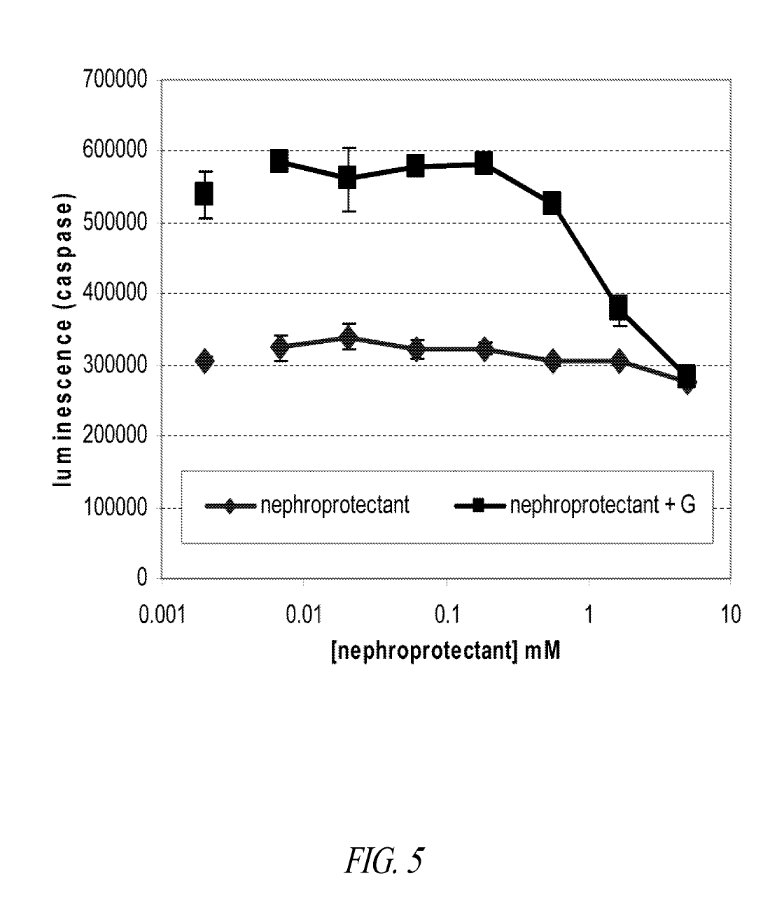

This example describes various counterscreens that may be used to validate candidate nephroprotectant compounds. Candidate nephroprotectants from an initial screen were subjected to one of more counterscreens to discard any that are interfering with the assay readout rather than inhibiting gentamicin-induced toxicity. A schematic of this process is shown in FIG. 6.

A trivial explanation for the reduction in caspase signal is that the additives themselves are toxic to the cells. This was ruled out by a standard viability assay (CELLTITERBLUE, Promega) run at the same concentrations as the initial screen. The CASPASEGLO reporter used in the in vitro assay provided a masked luciferase substrate that was activated by caspase enzyme present in apoptotic cells. The activated substrate was then available to a luciferase enzyme which was also included in the reporter kit. The combination of caspase and luciferase activities generated the luminescence signal that was read in the assay. Inhibition of either of these enzymes by putative nephroprotectants would yield a false positive in the screen.

Although compounds that inhibit luciferase are rare, a second counterscreen was employed to eliminate these using CELLTITERGLO (Promega). This reagent requires only viable cells (not caspase enzyme from apoptotic cells) to generate luminescence.

Finally, compounds were tested for the ability to inhibit caspase in an in vitro enzyme assay. Caspase inhibitors may be of interest as general inhibitors of apoptosis that also serve to reduce nephrotoxicity, so this screen was used to identify these compounds, but they were not eliminated as candidate nephroprotectants.

Several compounds demonstrated a nephroprotective effect in the HK-2 assay without affecting cell viability or the luciferase readout in the assay. All of these compounds decreased the gentamicin induced apoptosis signal in the assay without altering the background signal. These results demonstrated that the aforementioned compounds were directly inhibiting aminoglycoside related nephrotoxicity and not a more general process.

The effect of these nephroprotectants on the gentamicin minimal inhibitory concentration (MIC) in vitro is determined. Compounds that do not raise the gentamicin MIC are considered particularly promising candidate nephroprotectants.

### Example 6

### In Vitro HK-2 Cell-Based Nephrotoxicity Assay Protocol

This example describes one specific protocol for determining nephrotoxicity of a compound according to the methods described herein.

### Procedure

A human kidney proximal tubule cell line, HK-2 (ATCC #CRL-2190) is cultured in Keratinocyte-Serum Free Medium (K-SFM) (Invitrogen 17005-042) with 5 ng/mL of human recombinant epidermal growth factor (EGF) and 0.05 mg/mL Bovine Pituitary Extract (BPE). Culture medium containing EGF and/or BPE is not filtered subsequent to growth factor addition, and fresh growth factors are added to the culture medium after two weeks. The HK-2 cells are split so that they are 80% confluent on the day they need to be plated into 96 well plates. These cells are not allowed to become confluent. The suggested schedule for passaging these cells is to split them 1-2 times per week at dilutions of 1:2 or 1:3.

### Day 1, Plate Cells

Remove the HK-2 cells from the plate using trypsin-EDTA. Wash the plate with D-PBS. Add 0.5 mL trypsin, and incubate for 1 minute at room temperature or until cells start to lift off plate. Inactivate trypsin and harvest cells by adding prewarmed culture medium (37° C.) to the plate. Pipet the cells with a P1000 to disassociate them, and count the cells, adjusting them to a concentration of 1.6×10⁵ cells/mL in culture medium. Dispense 100 μL/well (1.6×10⁴ cells/well) into 96 well plates (tissue culture treated white plate with clear bottom; Greiner EK-25098, E&K Scientific), and incubate the plates at 37° C. with 5% CO₂ for 3 days.

### Day 4, Morning, Compound Addition

Make dilutions of experimental compounds in prewarmed assay medium (37° C.) in dilution plates. Make at least seven 2-fold serial dilutions starting at 500 μg/mL. Compound dilutions can be made and stored at 4° C. for 1-4 days with a low evaporation lid and parafilm. Warm the compound dilution plate in incubator for 10-15 minutes prior to addition to cells. Remove the 96 well plates with HK-2 cells from incubator, and aspirate the medium from wells. Aliquot the warmed compounds into plates at a volume of 100 μL/well, and incubate the plates at 37° C. with 5% CO₂ overnight (˜31 hours).

### Day 5, Harvest

Thaw the CASPASEGLO reagents (CASPASEGLO 3/7 Assay, Promega G8093 10×10 mL or G8092 100 mL) at room temperature. For reagent addition to plates after 31 hour compound incubation: (i) remove plates from incubator (30.5 hours after adding compounds) for approximately 30 minutes to bring to room temperature; (ii) mix CASPASEGLO reagents according to manufacturer's instructions; (iii) add 50 μL of reagent/well to 96 well plates using BioMek®FX with tips 2 mm from bottom of reservoir well (Beckman Coulter); (iv) place plates on shaker platform and shake gently for 30 seconds; (v) incubate at room temperature for 30 minutes; (vi) place plates on shaker platform and shake gently for 30 seconds; and (vii) read plate 30-60 minutes after reagents are added, Analyst™ HT (LJL Biosystems™). The settings for the Analyst™ HT are: (i) default luminescence method; (ii) luminescence height, 1.5 mm; (iii) 5 readings per well; (iv) integration time of 100000 μs; and (v) attenuator out.

### Data Analysis

The average luminescence values are calculated along with the standard deviation of the replicates. The concentration of the aminoglycoside tested is graphed versus the corresponding luminescence reading. The area under the curve (AUC) is calculated for each experimental compound and for gentamicin. The EC50 can then be calculated for each experimental compound and gentamicin, using methods well known in the art.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method for determining the nephrotoxicity of one or more test compounds, said method comprising:
(i) contacting discrete populations of HK-2 cells with one or more test compounds;
wherein a plurality of different concentrations for each of said one or more test compounds contacts a separate discrete population of HK-2 cells;
(ii) determining the level of an indicator of nephrotoxicity for each of said populations of HK-2 cells contacted in step (i) to produce a dose response curve for each of the one or more test compounds; and
(iii) determining the nephrotoxicity of each of said one or more test compounds.

2. The method of claim 1, wherein each of the populations of HK-2 cells are located in separate wells in a tissue culture device comprising a plurality of wells.

3. The method of claim 1, further comprising:
(iv) contacting additional discrete populations of HK-2 cells with a plurality of concentrations of one or more control nephrotoxic compounds;
(v) determining the level of an indicator of nephrotoxicity for each of said additionally contacted populations of HK-2 cells contacted in step (iv) to produce a dose response curve for each of said one or more control nephrotoxic compounds; and
(vi) determining the nephrotoxicity of each of the one or more control nephrotoxic compounds.

4. The method of claim 3, wherein the plurality of concentrations of the one or more test compounds tested and the plurality of concentrations of the one or more control nephrotoxic compounds tested are in the range of about 1 mg/mL to about 1 ug/mL.

5. The method of claim 3, wherein the determination of the nephrotoxicity of each of said one or more compounds comprises calculating an EC50 for each of the one or more test compounds from the dose response curves of step (ii).

6. The method of claim 5, wherein the determination of the nephrotoxicity of each of said one or more compounds comprises comparing the EC50 for each of the one or more test compounds to an EC50 for each of the one or more nephrotoxic compounds calculated from the dose response curves from step (iv).

7. The method of claim 3, wherein the one or more control nephrotoxic compounds are selected from the group consisting of: amikacin, gentamicin, kanamycin, neomycin, netilmicin, paromomycin, streptomycin, tobramycin, apramycin.

8. The method of claim 1, wherein the one or more test compounds are aminoglycosides.

9. The method of claim 8, wherein the indicator of nephrotoxicity is apoptosis.

10. A method for determining the ability of one or more candidate nephroprotectant compounds to act as a nephroprotectant, said method comprising:
(i) contacting discrete populations of HK-2 cells with a plurality of different concentrations of one or more candidate nephroprotectant compounds in the presence of a nephrotoxic compound, wherein each of a plurality of different concentrations for each of said one or more candidate nephroprotectant compounds contacts a separate discrete population of HK-2 cells;
(ii) determining the levels of an indicator of nephrotoxicity for each one of the contacted populations of HK-2 cells of step (i); and
(iii) determining the ability of each one or more candidate nephroprotectant compounds to act as a nephroprotectant.

11. The method of claim 10, further comprising:
(iv) contacting additional discrete populations of HK-2 cells with a plurality of different concentrations of said one or more candidate nephroprotectant compounds in the absence of a nephrotoxic compound, wherein each of a plurality of different concentrations for each of said one or more candidate nephroprotectant compounds contacts a separate discrete population of HK-2 cells;
wherein the ability of a candidate nephroprotectant compound to act as a nephroprotect is determined when a dose-dependent decrease in the indicator of nephrotoxicity is present in the contacted HK-2 cells of step (i) compared to the indicator of nephrotoxicity in the contacted HK-2 cells of step (iii) for a given candidate nephroprotectant compound.

12. The method of claim 11, wherein the indicator of nephrotoxicity is apoptosis.

13. The method of claim 12, wherein the indicator of nephrotoxicity is caspase activity.

14. The method of claim 10, said method further comprising:
(iv) validating the ability of said one or more candidate nephroprotectant compounds to act as a nephroprotectant by performing one or more counterscreens.

15. The method of claim 14, wherein said one or more counterscreens are selected from the group consisting of: a cell viability assay, and an assay for luciferase activity.

16. The method of claim 15, wherein the counterscreens are a cell viability assay and an assay for luciferase activity.

17. The method of claim 16, wherein the luciferase activity is produced in the presence of viable cells.

18. The method of claim 16, wherein a decrease in the number of cells in the cell viability assay and/or a decrease in the luciferase activity determines that the one or more candidate nephroprotectant compounds are not nephroprotectants.

19. The method of claim 10, wherein the one or more candidate nephroprotectant compounds are selected from the group consisting of: antioxidants, compounds that structurally resemble antioxidants, peptides, peptidomimetics, amino acids, amino acid analogs, polynucleotides, polynucleotide analogs, nucleotides, nucleotide analogs, and organic, inorganic compounds, and any combination thereof.

20. A method for determining the nephrotoxicity of one or more test compounds, said method comprising:
(i) contacting discrete populations of LLC-PK1 cells with one or more test compounds;
wherein a plurality of different concentrations for each of said one or more test compounds contacts a separate discrete population of LLC-PK1 cells;
(ii) determining the level of an indicator of nephrotoxicity for each of said populations of LLC-PK1 cells contacted in step (i) to produce a dose response curve for each of the one or more test compounds, wherein said indicator measures luciferase activity;
(iii) determining the nephrotoxicity of each of said one or more test compounds;
(iv) contacting additional discrete populations of LLC-PK1 cells with a plurality of concentrations of one or more control nephrotoxic compounds;
(v) determining the level of an indicator of nephrotoxicity for each of said additionally contacted populations of LLC-PK1 cells contacted in step (iv) to produce a dose response curve for each of said one or more control nephrotoxic compounds; and
(vi) determining the nephrotoxicity of each of the one or more control nephrotoxic compounds;
wherein each of the populations of LLC-PK1 cells are located in separate wells in a tissue culture device comprising a plurality of wells.
